(19) United States
(12) Patent Application Publication
(10) Pub. No.: US 2024/0033151 A1
(43) Pub. Date: Feb. 1, 2024

(54) HEAD SUPPORT APPARATUS

(71) Applicant: INNOVGAS PTY LTD, Launceston (AU)

(72) Inventor: Andrew WALLIS, Launceston (AU)

(73) Assignee: INNOVGAS PTY LTD, Launceston (AU)

(21) Appl. No.: 18/258,340

(22) PCT Filed: Dec. 23, 2021
(86) PCT No.: PCT/AU2021/051559

§ 371 Date: Jun. 20, 2023

(30) Foreign Application Priority Data
Dec. 28, 2020 (AU) 2020904860

(51) Int. Cl. A61G 13/12

(52) U.S. Cl. CPC A61G 13/121; A61G 13/126; A61G 2200/325; A61G 2200/322; A61G 2200/327

(57) ABSTRACT

There is proposed a head support apparatus, comprising a frame and an adjustable sling member. The frame including a base and at least two spaced apart upwardly extending side walls, the base being supportable on a patient receiving surface. The adjustable sling member configured to be reversibly attached to a head of a patient, wherein the sling member being reversibly couplable to the upwardly extending side walls of the frame to thereby support the head of the patient above the patient receiving surface. This allows a practitioner to check the patients eyes for anything pressing on them and to check the breathing tube for position, circuit disconnection, kinking or blockages, even when the patient is positioned in the prone orientation.

### FIELD OF THE INVENTION

The present invention relates to a method and apparatus for supporting the head of a patient while positioned on an operating table or hospital bed, and in particular to the support of a patient's head when in a prone orientation.

### BACKGROUND OF THE INVENTION

During surgical procedures and in other medical situations, it may be necessary to support the head of a patient. This is particularly the case when a patient is in a prone orientation, wherein they are chest down on a bed or operating table, with their dorsal side facing upwards. There may also be situations when a patient's head requires support when they are positioned in a supine orientation, a left lateral recumbent orientation or a right lateral recumbent orientation.

There are a number of prone position head rests currently in use or suggested in the published prior art. One such prior art device comprises a block of single use foam that includes a void or voids being configured to align the eyes, nose and mouth of a patient. The forehead and chin of the patient is supported on the top of the foam block, comprising at least two layers of different density foam material.

However, the configuration of the existing foam blocks may obscure an airway device of an anaesthesia circuit or inhibit access or observation of the patient's eyes, because the cut outs may not accurately fit the shape or size of the patient's face. Furthermore, the existing foam blocks tend to wrap around and conform to the patient's face thereby further reducing access. These devices do not typically allow for the dynamic adjustment in patient head height.

There are also moulded prone position support devices on the market that are constructed from silicone or rubber compounds. These include anatomically specific contouring to engage the face of the patient. Although such devices can be used multiple times, the moulded nature of the devices means that they can only be used for a limited range of face shapes and sizes. This means that different dimensioned devices must be manufactured and be available for use with different patients.

There are also prone position head supports that are adjustable and include movable head cushion portions. Such devices may include a frame that is supportable on a bed surface, a forehead support cushion and a chin support cushion. The forehead support cushion and/or chin support cushion slidably engage the frame such that the head support device can be adjusted to accommodate different sized heads. The cushioned portions are however moulded and may not accurately fit the patient's face, leading to pressure lesions or abrasion.

There are also facial cradle devices, such as the device disclosed in U.S. Pat. No. 9,867,749 (Mitchell), which includes a facial cradle for facial support of a prone patient's head. The facial cradle is configured to be supported by arms that extend outwardly from an end of a specialised table on which a patient is positioned.

One of the limitations with existing prone position devices is that the patient's head only engages the device when they are in the prone orientation. This means that the head is not supported during rolling of a patient between a supine orientation and a prone orientation. This can pose issues when the patient is anesthetised or otherwise unconscious, and for older patients, or those with neck injuries.

It should be appreciated that any discussion of the prior art throughout the specification is included solely for the purpose of providing a context for the present invention and should in no way be considered as an admission that such prior art was widely known or formed part of the common general knowledge in the field as it existed before the priority date of the application.

The reader will appreciate that the apparatus is discussed with particular reference to the support of a patient's head when in the prone orientation, but the reader should appreciate that the apparatus may be used for a patient in the supine orientation or lateral recumbent orientations. The terms “bed”, “table” or similar, used throughout the specification should be given its broadest definition and refers to the surface upon which the patient is positioned.

### SUMMARY OF THE INVENTION

It is an object of at least some of the illustrated embodiments to provide for a prone position head support for a patient which conforms to the face of the patient, while permitting access to the eye region or mouth/nose regions of the patient's face. It is a further object of the illustrated embodiments to overcome at least some of the aforementioned problems, or at least provide the public with a useful alternative. The foregoing objects should not necessarily be considered as cumulative and various aspects of the invention may fulfil one or more of the above objects.

Broadly the invention could be understood to comprise a head support apparatus including a frame and an adjustable sling member being reversibly attachable to the frame, wherein the adjustable sling member is fastenable to the head of a patient, whereafter the adjustable sling member is reversibly attached between upwardly extending portions of the frame to thereby support the head of the patient.

In one aspect of the invention, but not necessarily the broadest or only aspect, there is proposed a patient head support apparatus, comprising: a frame including a base and at least two spaced apart upwardly extending side walls, the base being supportable on a patient receiving surface; and an adjustable sling member configured to be reversibly attached to a head of the patient, wherein the sling member being reversibly couplable to the upwardly extending side walls of the frame to thereby support the head of the patient above the patient receiving surface.

The patient receiving surface may be a hospital bed, operating table or other surface upon which the patient may be placed.

Preferably, the patient head support apparatus is used for a patient in a prone orientation, however the reader should appreciate that the apparatus is not limited to such use, and the apparatus may support the head of a patient in a supine orientation, a left lateral recumbent orientation, a right lateral recumbent orientation or a position therebetween.

The frame, in one form, includes a generally rigid base having two spaced apart side walls extending upwardly from edges thereof. The frame may comprise two interconnectable generally identical portions that can be connected together to form the base and side walls. Alternatively, the frame may be unitary in construction.

The frame may be injection moulded or blow-moulded and may be constructed from a plastics material, such as but not limited to, cross-linked polyethylene (PEX or XLPE), polyvinyl chloride (PVC), high density polyethylene (HDPE) or chlorinated polyvinyl chlorine (CPVC).

The side walls may include respective voids extending therethrough, which can be used by a practitioner to grasp when repositioning the apparatus when the patient's head is supported thereon.

The adjustable sling member, in one form, includes a head/face abutment portion, adjustment straps, a head securing member, handles and couplings.

The head/face abutment portion may comprise a resilient deformable pad that is configured to conform to the unique profile of the patient's head or face. In a preferred form, when the patient's head is to be supported in the prone orientation, the head/face abutment portion includes a void or voids extending therethrough, which are positionable adjacent to the eye region and/or mouth/nose regions of the patient's face. This means that the eyes and mouth/nose regions are visible and/or accessible throughout a medical procedure.

In one form, the void corresponding to the eye region of the patient, is dimensioned to accommodate the eye cover previously disclosed by the present Applicant in International Application, PCT/AU2015/050481 entitled SURGICAL EYE SHIELD, which is incorporated herein by reference.

The resilient deformable pad may be constructed from polyurethane foam, latex foam, or any other type of material that is suitable to provide adequate support for a patient's head or face. In one form, the resilient deformable pad may be reversibly couplable to the adjustable sling member, such that it can be replaced between patients. Alternatively, the resilient deformable pad may be protected by a replaceable cover, or the adjustable sling member may be disposed after a single use.

The adjustment straps, in one form, are configured to be fixedly connected to the head/face abutment portion and adjustably connected through respective apertures in the handles. In one form the adjustment straps may include hook and loop fastener portions, to facilitate adjustment. Alternatively, buckles could be used or any other type of suitable temporary fixing means.

The head securing member may likewise comprise a band including hook and loop fastener portions, wherein the band can be secured around the back of the patient's head to hold the adjustable sling member in place. In this way the adjustable sling member is secured around the patient's head independent of the frame.

In use the adjustable sling member is secured to the head of the patient when they are in a supine orientation. The patient can then be moved into the prone orientation on the hospital bed or operating table, whereafter the adjustable sling member is attached to the side walls of the frame, using respective couplings, to thereby hold the head of the patient in place.

When the patient is first turned into the prone orientation there is usually further adjustment of supports on the bed e.g. pillows, whereby the patient's body is slid, twisted or tilted for optimal positioning. The handles therefore allow the user to support the patient's head while this is being carried out. This is especially important for larger patients, where due to their size, optimal positioning can be difficult.

The handles of the adjustable sling member can be used to support and manipulate the head of the patient whilst they are being moved and can be utilised to assist in positioning the head in place relative to the frame.

Accordingly, the adjustable sling member can be used by a practitioner independently of the frame to support the head while the patient is being moved. The adjustable sling member can then be clipped or otherwise coupled, onto the frame to thereby support the head during a medical procedure.

Preferably, each handle incorporates a respective coupling which is configured to engage an upper edge of a respective side wall of the frame. In this way the handles can be clipped onto, and removed from, the side walls of the frame. Alternatively, respective couplings can be separate from and adjacent each of the handles.

Once the adjustable sling member has been attached to the frame the adjustment straps can be used to modify the orientation of the patient's head to ensure it is adequately supported whilst allowing access, either physical or visual, to the eye region and mouth/nose regions. This allows a practitioner to check the patient's eyes for anything pressing on them and to check the breathing tube for position, circuit disconnection, kinking or blockages.

Preferably, a first adjustment strap extends across or adjacent the forehead region of the patient between the spaced apart side walls of the frame, and a second adjustment strap extends across or adjacent the chin region of the patient between the spaced apart side walls.

When the surgical or medical procedure is completed the handles can be grasped by a practitioner and manipulated to unclip the adjustable sling member from the side walls of the frame. The frame can then be removed and the patient transferred onto a gurney or other surface.

In the situation that the patient has been orientated in the prone orientation, they can be rolled back into the supine orientation, whereafter the adjustable sling member can be removed from the head of the patient. The reader will appreciate that the handles assist a practitioner in controlling the movement of the patient's head when they are being moved or rolled.

The adjustable sling member is preferably, at least partly flexible and configured to conform to a range of head sizes and shapes. Preferably, the frame is generally rigid in construction. In this way the flexible sling is reversibly couplable to upper ends of spaced apart portions of the generally rigid frame, whereby at least a part of the head of the patient is supported within the flexible sling and extends below the upper ends of the spaced apart portions of the generally rigid frame.

In one form, a sensor or sensors may be embedded into, or be attached on a face engaging surface of, the adjustable sling member. The sensor or sensors may be used to monitor the dynamic pressure changes throughout the operation and provide a practitioner with information that will enable them to make decisions regarding patent care, including the readjustment or temporary removal of the adjustable sling member. The readjustment or temporary removal of the adjustable sling member may be beneficial in minimising skin pressure lesions, postoperative pain and/or neural damage.

The apparatus may further include a controller, CPU and display unit in communication with the sensor or sensors. These will not be described in detail since they would be obvious to a person skilled in the art.

In still another aspect of the invention there is proposed a method of supporting the head of a patient in a prone orientation, including the steps of:
providing a head support apparatus as hereinbefore described;
positioning the patient in a supine orientation;
attaching an adjustable sling member of head support apparatus to the head of the patient, wherein an abutment portion of the adjustable sling member abuts the face of the patient and a void or voids extending through the abutment portion permits visual observation of, or access to, the patient's eye region and/or mouth/nose regions;
rolling or otherwise moving the patient into the prone orientation, wherein handles of the adjustable sling member are utilised to support the head of the patient, and permitting further adjustment of the patient's body and limbs while supporting the patient's head;
positioning a frame of head support apparatus below and adjacent the face of the patient;
attaching the adjustable sling member to the side walls of the frame wherein the patient's head is spaced apart from a surface on which the patient is positioned; and adjusting straps of the adjustable sling member to ensure appropriate head height and angle relative to the patient's body, while providing adequate support of the patient's head.

### BRIEF DESCRIPTION OF THE DRAWINGS

The accompanying drawings, which are incorporated in and constitute a part of this specification, illustrate an implementation of the invention and, together with the description and claims, serve to explain the advantages and principles of the invention. In the drawings,

FIG. 1 is a perspective view of the head support apparatus, comprising a frame and an adjustable sling member attached thereto;

FIG. 2 is an end view of the head support apparatus of FIG. 1;

FIG. 3 is a side view of the head support apparatus of FIG. 1;

FIG. 4 is a top view of the head support apparatus of FIG. 1, illustrating the adjustable sling member detached from the frame;

FIG. 5 is a side view of the head support apparatus of FIG. 1, illustrating the support of a patient's head;

FIG. 6 is a rear perspective view of FIG. 5;

FIG. 7 is a top view of the frame of FIG. 1, illustrating the separate portions that are attachable to thereby form the frame;

FIG. 8 is a partial view of one of the side walls of FIG. 1, illustrating the attachment of the handle;

FIG. 9 is a partial view of the handle of FIG. 8;

FIG. 10 is a perspective view of the handle of FIG. 8;

FIG. 11 is a perspective view of the head support apparatus of FIG. 1, illustrating the adjustment of one of the straps, without a patient's head shown;

FIG. 12 is a perspective view of the head support apparatus of FIG. 11, illustrating the attachment of the strap to a fastener portion on the handle;

FIG. 13 is a perspective view of the head support apparatus of FIG. 11, illustrating the removal of the strap from the fastener portion on the handle for further adjustment;

FIG. 14 is a perspective view of the head support apparatus of FIG. 11, illustrating all straps attached to respective fastener portions;

FIG. 15 is a side view illustrating the head support apparatus of FIG. 14, with a patient's head in position;

FIG. 16 is a perspective view of another embodiment of the head support apparatus, illustrating the adjustable sling member detached from the frame;

FIG. 17 is a perspective view of the head support apparatus of FIG. 16, illustrating the adjustable sling member attached to the frame;

FIG. 18 is a perspective view of the head support apparatus of FIG. 17, illustrating the head of a patient supported thereon; and

FIG. 19 is a perspective view of the frame of FIG. 16 illustrating the separate portions that are attachable to thereby form the rigid frame.

### DETAILED DESCRIPTION OF THE ILLUSTRATED AND EXEMPLIFIED EMBODIMENTS

Similar reference characters indicate corresponding parts throughout the drawings. Dimensions of certain parts shown in the drawings may have been modified and/or exaggerated for the purposes of clarity or illustration.

Referring to the drawings for a more detailed description, there is illustrated a head support apparatus 10, demonstrating by way of examples, arrangements in which the principles of the present invention may be employed.

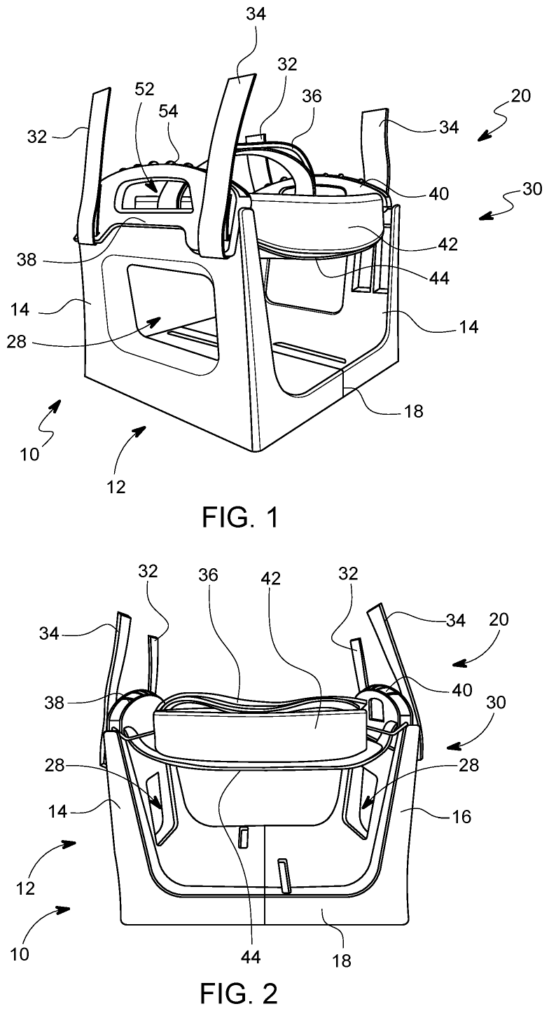

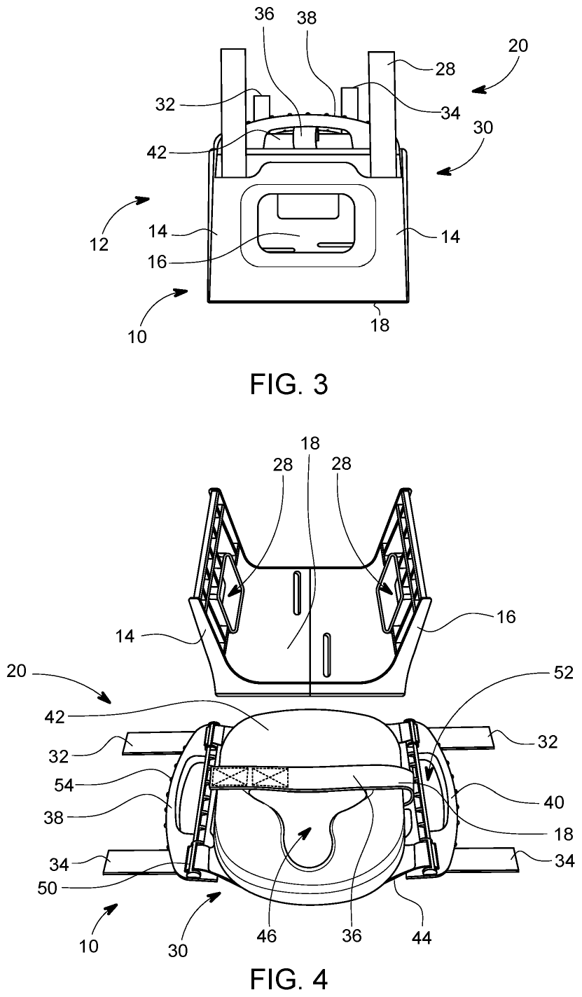

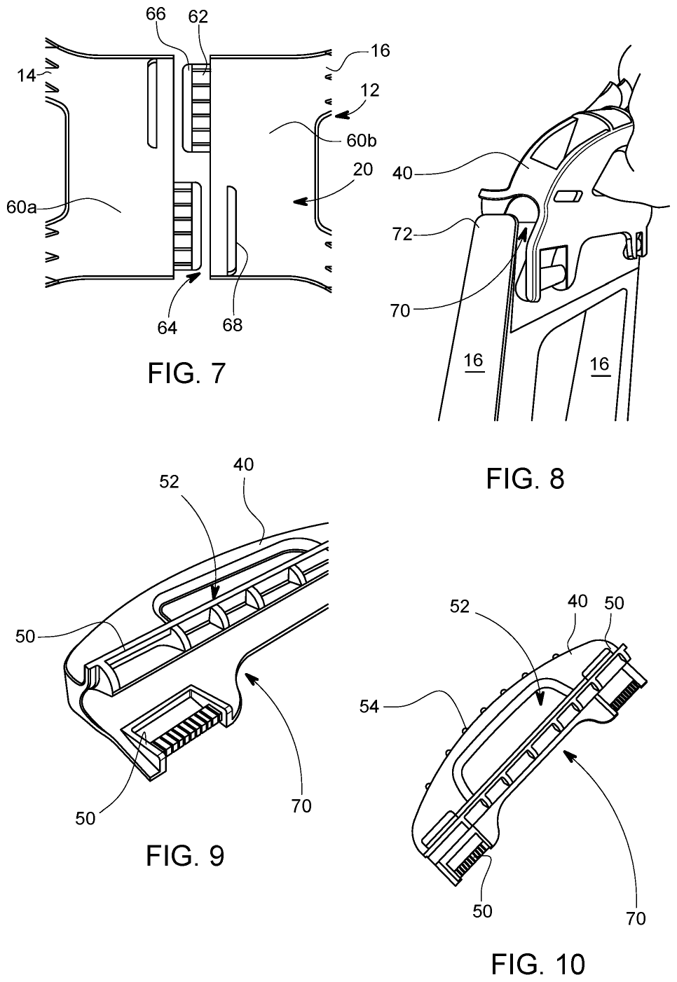

As illustrated in FIGS. 1 to 4, the head support apparatus 10, includes a frame 12 having spaced apart upwardly extending side walls 14, 16, and a base 18. The frame 12 may comprise two generally identical portions that can be attached together to form the base and side walls, as illustrated in FIGS. 7 and 19. This can assist in transport and/or manufacture of the apparatus 10, however the reader should appreciate that the base and side walls may be unitary in construction.

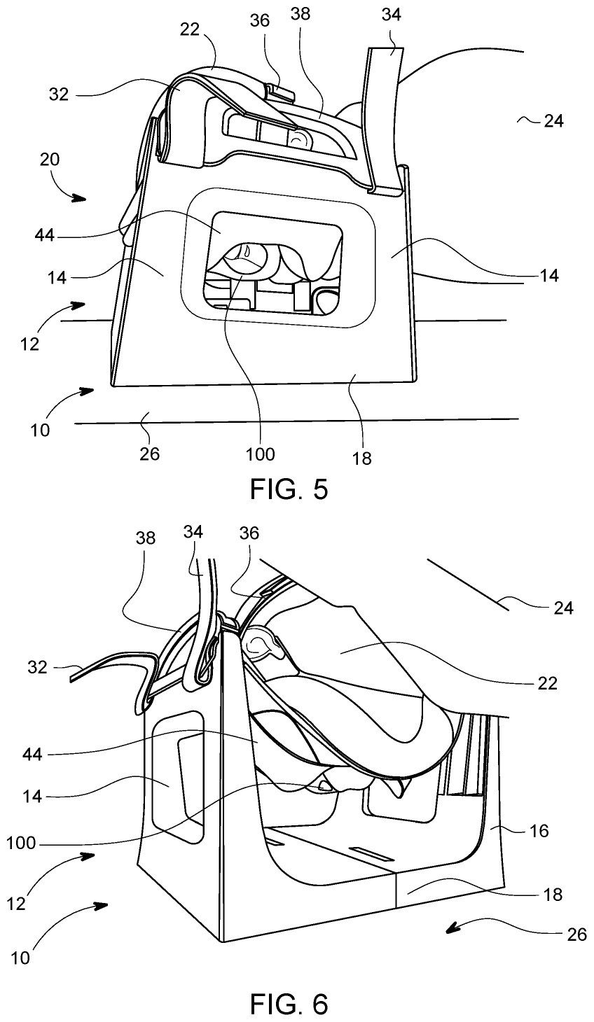

The apparatus 10 further comprises an adjustable sling member 20 configured to engage a head 22 of a patient 24, being positioned on a patient receiving surface 26, such as an operating table, as illustrated in FIGS. 5 and 6.

The sling member 20 is reversibly attachable to the upwardly extending side walls 14, 16 to thereby support the head of the patient 24, in the prone orientation, as illustrated in the figures. However, the skilled addressee will appreciate that the patient 24 can be supported in the supine orientation or right/left lateral recumbent orientations.

The side walls 14, 16 include respective voids 28 that extend therethrough, which can be grasped by a practitioner and used to assist in repositioning the apparatus 10, when the patient's head 22 is supported thereon. The voids 28 can also be used when the frame 12 is being positioned below the patient's face or removed after use.

The adjustable sling member 20 includes an abutment portion 30, adjustment straps 32, 34, a head securing band 36, handles 38, 40, and couplings 70.

The abutment portion 30 comprises a resilient deformable pad 42 and a backing 44 attached thereto. The backing 44 is fixedly connected to, or forms part of, the adjustment straps 32, 34 and head securing band 36.

As illustrated in FIG. 4, the resilient deformable pad 42 includes a void 46 extending therethrough, which is positionable relative to the eyes and mouth/nose of the patient 22, as shown in FIGS. 5 and 6. This means that the eyes and mouth/nose regions of the patient are visible and/or accessible during use of the apparatus 10. The reader will however appreciate that multiple voids may be used rather than the single generally T-shaped void, as illustrated in the figures.

As further illustrated in FIGS. 5 and 6, the portion of the void 46 corresponding to the eye region of the patient 24, is dimensioned to accommodate the eye cover 100 previously disclosed by the present Applicant in International Application, PCT/AU2015/050481 entitled SURGICAL EYE SHIELD, which is incorporated herein by reference.

In some embodiments, the surgical eye shield 100 or similar device is configured to register with the void 46 which thereby assists in the correct fitting of the adjustable sling member 20 and maintaining its position relative to the face when the patient is being moved or during the medical procedure. The surgical eye shield 100 or similar device is attached to the face of the patient and therefore holds the resilient deformable pad 42, and hence the adjustable sling member 20 in place during use.

In one form, the surgical eye shield 100 or similar device is shaped to frictionally engage the edge of the void 46 through the resilient deformable pad 42. This means that the visual access to the patient's eyes is maintained, while inhibiting impact which may occur to the patient's eyes during the medical procedure.

Preferably, the adjustment strap/s 32 extends across, or adjacent the forehead region of the patient and the adjustment strap/s 34 extends across, or adjacent the chin region of the patient between the spaced apart side walls 14, 16 of the frame 12.

The handles 38, 40 are adjustably couplable to the adjustment straps 32, 34. In the present embodiment, the adjustment straps 32, 34 extend through respective slots 50 in the handles 38, 40 to facilitate the adjustment.

The handles 38, 40 further include respective voids 52 and grips 54 to assist a practitioner with the grasping thereof.

As illustrated in FIGS. 7 and 19, the frame 12 comprises two generally identical portions 60a, 60b, each including a tongue 62 that is configured to engage a respective passageway 64. Each tongue 62 includes hooks 66 that are adapted to engage a respective slot 68, which adjoin a corresponding passageway 64.

As illustrated in FIGS. 8 to 10, the handle 40 in the present embodiment incorporate coupling 70 in the form of a downwardly open channel. The downwardly open channel is configured to engage a correspondingly shaped upper edge 72 of the side wall 16. The reader will appreciate that handle 38 also includes a downwardly open channel 70, which is configured to engage a correspondingly shaped upper edge 72 of the side wall 14, as illustrated in FIG. 16.

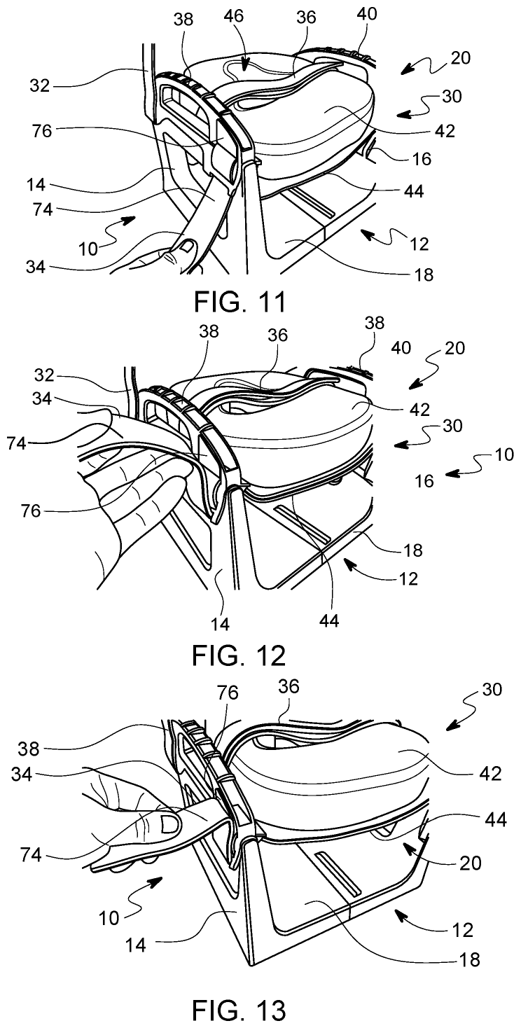

FIGS. 11 to 15, illustrate the adjustment of the adjustable sling member 20. The adjustment straps 32, 34, and head securing band 36 include respective hook and loop fastener portions for adjustment thereof. FIG. 11 illustrates the strap 34, having a loop portion 74, detached from the corresponding hook portion 76 on the handle 38. The reader will appreciate that this also applies to the straps 32 and attachment to handle 40. Furthermore, it should be appreciated that FIGS. 11 to 14 do not illustrate the patient's head 22 so as to not obscure the features of the apparatus 10.

FIG. 12 illustrates the attachment of the loop portion 74 of the strap 34, to a fastener portion 76 on the handle 38. FIG. 13 illustrates the removal of the strap 34 from the fastener portion 76 on the handle 38 to enable further adjustment of the adjustable sling member 20.

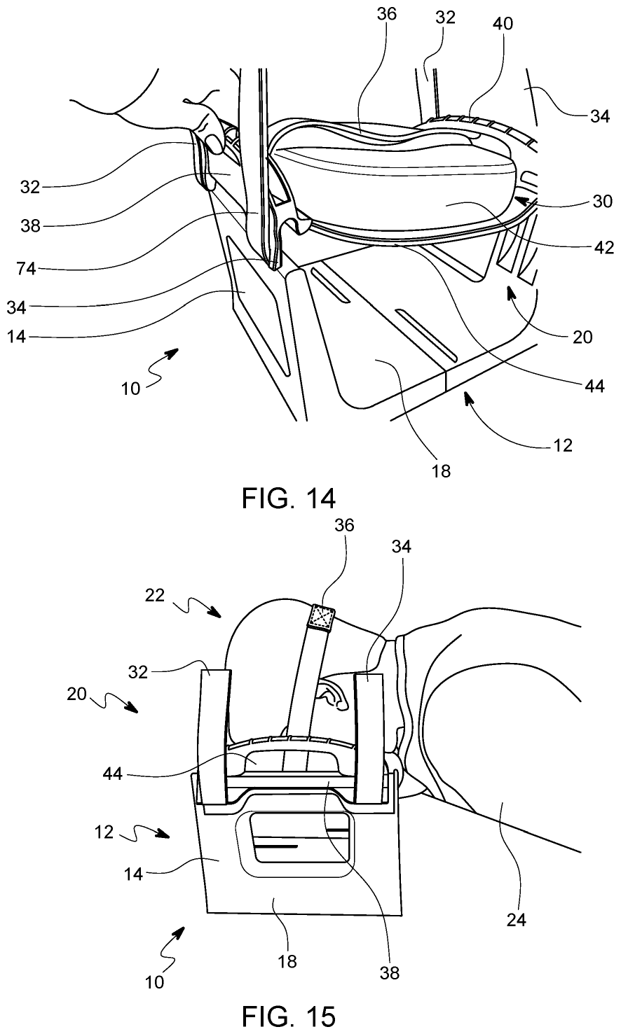

FIGS. 14 and 15 illustrate the attachment of the straps 32, 34 to thereby support the head 22 of the patient 24. The reader should appreciate that the fine adjustment of the adjustable sling member 20 is undertaken when the head 22 of the patient 24 is being supported thereon.

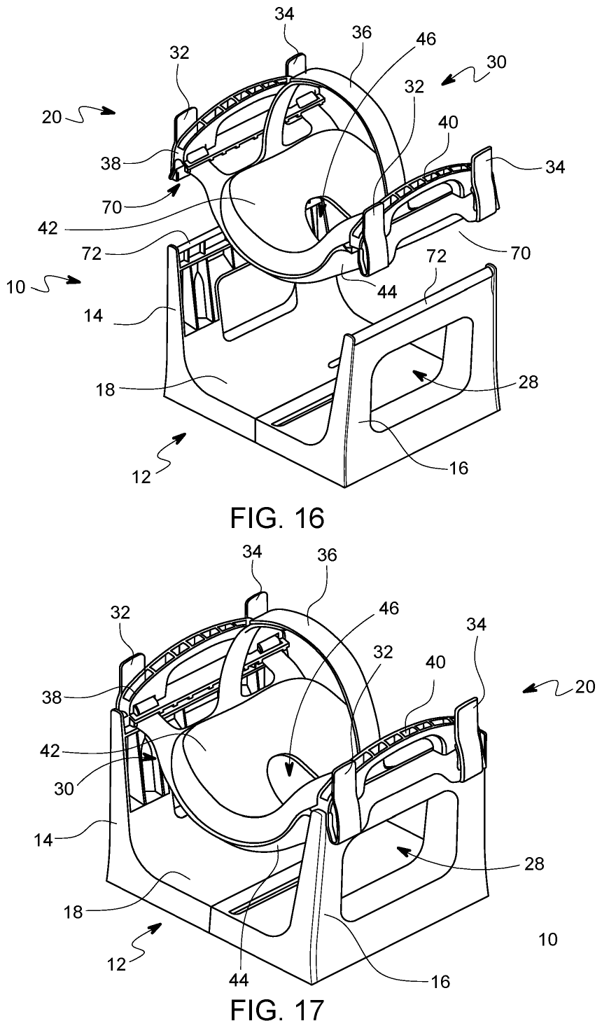

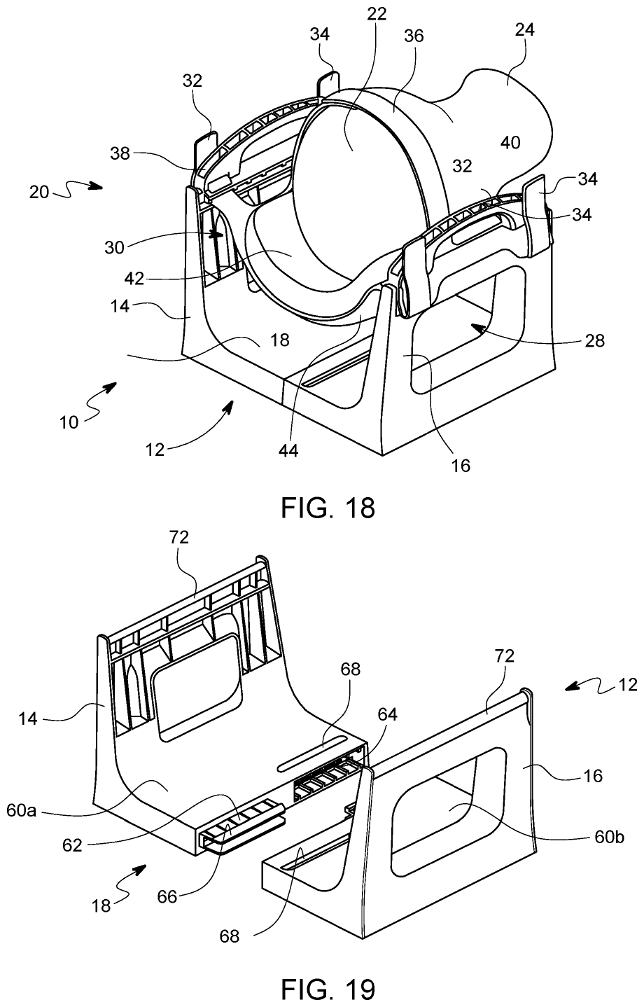

In another embodiment, as illustrated in FIGS. 16 to 19, the resilient deformable pad 42, may be partially moulded or shaped to generally conform to the face of the patient 24. The resilient deformable pad 42 may also include different layers of foam material to provide varying degrees of comfort and support or may be constructed from a single density foam material.

The skilled addressee will appreciate that one advantages with the apparatus is that the adjustable sling member 20 can be attached to the head of the patient 24 while they are positioned in the supine orientation. The patient 24 can then be rolled into a prone orientation on the hospital bed or operating table 26 and the adjustable sling member 20 can then be attached to the frame 12 using coupling 70, to thereby support the head 22 of the patient 24. Fine adjustment of the adjustable sling member 20 can then be undertaken as previously discussed with respect to FIGS. 11 to 15 to ensure appropriate head height and angle relative to the patient's body, while providing adequate support and comfort for the patient 24, to minimise postoperative pain and/or neural damage.

The abutment portion 30 of the adjustable sling member 20 abuts the face of the patient and the void 46 extending through the abutment portion 30, permit visual observation of, or access to, the patient's eye region, and/or mouth/nose regions during a medical procedure. Because the patient's head is spaced apart from and above the table surface, a mirror (not shown) may also be positioned on top of the base 18 to assist in observing the patient's face during the procedure.

The skilled addressee will also appreciate that the handles 38, 40 can be used to support the head 22 of the patient 24 whilst they are being rolled or moved into position.

Although not illustrated the practitioner may use the handles 38, 40 to hold the head 22 of the patient 24, above the table surface 26 and the frame 12 can then be slid into position below the face of the patient 24. The handles 38, 40 can then be clipped onto the top of the frame 12.

When the surgical or medical procedure is completed the handles 38, 40 can be grasped by a practitioner and manipulated to unclip the adjustable sling member from the frame 12. The frame 12 can then be removed and the patient 24 transferred onto a gurney or other surface. In the situation that the patient 24 has been orientated in the prone orientation, they can be rolled back into the supine orientation, whereafter the adjustable sling member 20 can be removed from around the head 22 of the patient 24.

The skilled addressee will now appreciate the advantages of the illustrated invention over the prior art. In one form, the apparatus is configured to include at least two parts, wherein a first part, being the adjustable sling member 20, can be attached to the head of a patient prior to being positioned on an operation table 26 or when they are positioned on their back. Once the patient is positioned on the operation table 26 or when they are rolled into their front or side, the first part is couplable to a second part, being the frame 12. In this way the head is held in the adjustable sling or cradle during the operation or medical procedure.

The sling or cradle is able to conform to the patients face or head and can therefore accommodate a greater range of different sized and shaped faces compared to the prior art devices without physically or visually obstruction the eye or nose/mouth regions of the patient's face. The use of handles on the sling or cradle also means that a practitioner has greater control of the patient's head while they are being rolled and/or moved between a gurney and an operating table.

In the claims which follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

Various features of the invention have been particularly shown and described in connection with the exemplified embodiments of the invention, however it must be understood that these particular arrangements merely illustrate the invention and it is not limited thereto. Accordingly, the invention can include various modifications, which fall within the spirit and scope of the invention.

1. A patient head support apparatus, comprising:
a frame including a base and at least two spaced apart upwardly extending side walls, the base being adapted to be supported on a patient receiving surface; and
an adjustable sling member configured to be reversibly attached to a head of the patient, wherein the sling member is reversibly couplable to the upwardly extending side walls of the frame to thereby support the head of the patient above the patient receiving surface.

2. The patient head support apparatus in accordance with claim 1, being configured to support the head of said patient positioned in a prone orientation.

3. The patient head support apparatus in accordance with claim 1, being configured to support the head of said patient positioned in a supine orientation, a left lateral recumbent orientation, or a right lateral recumbent orientation.

4. The patient head support apparatus in accordance with claim 1, wherein the frame includes a generally rigid base, having two spaced apart generally rigid side walls extending upwardly from edges thereof.

5. The patient head support apparatus in accordance with claim 4, wherein the frame comprises two interconnectable portions being connectable to form the base and side walls.

6. The patient head support apparatus in accordance with claim 5, wherein the side walls include respective voids extending therethrough, wherein the voids are graspable by a practitioner for repositioning said patient head support apparatus when the patient's head is supported thereon.

7. The patient head support apparatus in accordance with claim 1, wherein the adjustable sling member includes a head/face abutment portion, adjustment straps, a head securing member, handles, and couplings.

8. The patient head support apparatus in accordance with claim 7, wherein the head/face abutment portion comprises a resilient deformable pad being configured to conform to a unique profile of the patient's head or face.

9. The patient head support apparatus in accordance with claim 8, wherein the head/face abutment portion includes a void or voids extending therethrough that are positionable relative to the eyes and mouth/nose regions of the patient's face, whereby the eyes and mouth/nose regions of the patent patient are visible or accessible throughout a medical procedure or operation, when the patient is positioned in a prone orientation.

10. The patient head support apparatus in accordance with claim 8, wherein the resilient deformable pad is reversibly couplable to the adjustable sling member.

11. The patient head support apparatus in accordance with claim 7, wherein the adjustment straps are configured to be fixedly connected to the head/face abutment portion and adjustable connected through respective apertures in a respective handle, and wherein the adjustment straps are used to modify the position of the patient's head once the adjustable sling member has been attached to the frame, to thereby ensure the patient's head is adequately supported whilst allowing access, either physical or visual, to the eye region and/or mouth/nose regions of the patient.

12. The patient head support apparatus in accordance with claim 7, wherein each handle incorporates a respective coupling which is configured to engage an upper edge of a respective side wall of the frame, whereby each handle is reversibly couplable to a respective side wall of the frame.

13. The patient head support apparatus in accordance with claim 11, wherein a first adjustment strap is adapted to extend across or adjacent the forehead region of the patient between the spaced apart side walls of the frame, and a second adjustment strap is adapted to extend across or adjacent the chin region of the patient between the spaced apart side walls.

14. The patient head support apparatus in accordance with claim 9, wherein a void of the patient head support apparatus is configured to register with a surgical eye shield reversibly affixable to the patient's face, to thereby assists in the correct fitting of the adjustable sling member and maintaining the adjustable sling member's position relative to the face when the patient is being moved or during the medical procedure or operation.

15. The patient head support apparatus in accordance with claim 1, wherein at least one sensor is embedded into, or is attached to a face engaging surface of the adjustable sling member for monitoring dynamic pressure changes throughout a medical procedure or operation.

16. A method of supporting the head of a patient in a prone orientation, including the steps of:
providing a head support apparatus in accordance with claim 1;
positioning the patient in a supine orientation;
attaching an adjustable sling member of the head support apparatus to the head of the patient, wherein an abutment portion of the adjustable sling member abuts the face of the patient and a void or voids extending through the abutment portion permits visual observation of, or access to, the patient's eye region and/or mouth/nose regions;
rolling or otherwise moving the patient into the prone orientation, wherein handles of the adjustable sling member are utilised to support the head of the patient, and permitting further adjustment of the patient's body and limbs while supporting the patient's head;
positioning a frame of head support apparatus below and adjacent the face of the patient;
attaching the adjustable sling member to the side walls of the frame wherein the patient's head is spaced apart from a surface on which the patient is positioned; and
adjusting straps of the adjustable sling member to ensure appropriate head height and angle relative to the patient's body, while providing adequate support of the patient's head.
